(19) United States
(12) Patent Application Publication
(10) Pub. No.: US 2026/0294851 A1
(43) Pub. Date: Oct. 1, 2026

(54) HYDROGEL PATCH FOR WOUND HEALING PROMOTION OR SCAR MINIMIZATION

(71) Applicants: CK REGEON INC., Seoul (KR); UIF (UNIVERSITY INDUSTRY FOUNDATION), YONSEI UNIVERSITY, Seoul (KR)

(72) Inventors: Kang-Yell CHOI, Seoul (KR); Soung-Hoon LEE, Seoul (KR); Min-Jeong KANG, Incheon (KR); Seung-Woo CHO, Seoul (KR); Soohwan AN, Seoul (KR)

(73) Assignees: CK REGEON INC., Seoul (KR); UIF (UNIVERSITY INDUSTRY FOUNDATION), YONSEI UNIVERSITY, Seoul (KR)

(21) Appl. No.: 19/100,878

(22) PCT Filed: Aug. 4, 2023
(86) PCT No.: PCT/KR2023/011505

§ 371 Date: Feb. 3, 2025

(30) Foreign Application Priority Data
Aug. 5, 2022 (KR) 10-2022-0097714

(51) Int. Cl. A61K 31/19; A61K 9/70; A61K 38/16; A61P 17/02

(52) U.S. Cl. CPC A61K 31/19; A61K 9/7069; A61K 38/16; A61P 17/02

(57) ABSTRACT

The present disclosure relates to a hydrogel patch for promoting wound healing or minimizing scarring. Since the hydrogel patch contains a pyrogallol-modified biocompatible polymer loaded with PTD-DMB, or a combination of PTD-DBM and valproic acid, it increases the content of collagen III, which is important for regenerative wound healing and scar reduction, and enhances the expression of the dermal stem cell marker CD105 and the angiogenesis marker CD31 at a wound site. Therefore, it can promote wound healing and minimize scarring.

### TECHNICAL FIELD

The present disclosure relates to a therapeutic hydrogel patch that can promote wound healing and minimize scarring, and a cosmetic pack containing the same.

### BACKGROUND ART

Skin is responsible for many functions in the human body. The most important function is to act as a barrier to protect the body from the outside. A cut or wound refers to the disruption of the skin's normal structure, which serves as a protective barrier.

When the skin is wounded, the wounded area is exposed and vulnerable to bacterial invasion. Therefore, prompt wound care is essential to prevent further infection and prevent excessive inflammation. Excessive inflammation not only prolongs the wound healing process, but also causes scarring.

Wound healing is clinically defined as the complete closure of the skin. A typical non-chronic wound heals completely in about 3-14 days. Wound healing is a complex process that involves the interaction and regulation by many cell types, including keratinocytes, fibroblasts, endothelial cells, macrophages, platelets, etc.

Currently, the main wound treatment drugs used in Korea are Dongkook Pharmaceutical's Madecasol (released in 1985) and Donghwa Pharm's Fusidine (released in 1980). While the efficacy of the two drugs is generally similar, Fusidine has antibacterial properties and Madecassol is slightly stronger at reducing scarring.

Although the existing steroid ointments are effective in terms of anti-inflammatory actions, immunosuppressive actions, treatment of allergic conditions, etc., they cause side effects such as pockmarks, skin wrinkles, folliculitis, etc., and are not effective against bacterial conditions such as athlete's foot. In addition, ointments containing antibiotics can develop resistance and cause a number of side effects when used on wounds of delicate skin, such as in children.

Therefore, wound care products based on the mechanism of wound healing are required to overcome the problems of steroid ointments or antibiotic ointments.

In addition, accidental injuries in daily life which are more severe than skin wounds, skin diseases such as melanoma, large wounds after surgery, and wounds associated with other conditions such as diabetes, etc. not only take time to heal, but often cause scarring. Daewoong Pharmaceutical released ‘EGF Saesal Ointment’, which is a wound treatment containing the epidermal growth factor (EGF), for the first time in Korea as an over-the-counter product. The epithelial cell growth factor helps wounds heal by covering the skin at the wound site (re-epithelialization) and promoting granulation (granulation tissue proliferation). However, because the epidermal growth factor is a protein, it requires very high cost and efforts for production, maintenance of safety, etc., and it does not have a significant impact on scarring.

On the other hand, in order to effectively repair tissues and organs damaged by a disease or accident, it is necessary to efficiently deliver drugs or cells in vivo or ex vivo. Therefore, a lot of research have been conducted on delivery of drugs or cells. However, the development of a system that can be used in clinical practice and shows satisfactory therapeutic effects is still insufficient.

Among the methods of drug delivery, the traditional administration methods such as oral administration has the issue of a short drug retention time due to rapid degradation and diffusion of the drugs and for effective treatment, it is necessary to inject a large amount of drugs or increase the frequency of drug injection.

Although researches have been conducted on a delivery method using hydrogels to solve the problems of the existing drug or cell delivery methods, most of them have involved loading of drugs or cells and crosslinking of them into hydrogel solutions to induce gel formation. However, since the above methods involve the injection of hydrogels into the body, it is likely that the results vary depending on the user's skill of handling the hydrogels, and the user's convenience is reduced greatly. In addition, because they are introduced into the body via injection, there is still a risk of tissue damage and bleeding, and the existing hydrogels often lack the physical properties and adhesiveness to be sustained in vivo for long periods of time.

To overcome the aforementioned limitations of the prior art, the inventors of the present disclosure have prepared a patch that has excellent drug delivery efficiency and user convenience due to the use of a pyrogallol-modified hyaluronic acid derivative and also is advantageous for clinical application without the need of an oxidant, wherein a drug with excellent efficacy for wound healing or scar reduction is loaded in a hydrogel.

### DISCLOSURE

### Technical Problem

The present disclosure is directed to providing a therapeutic hydrogel patch that can promote wound healing and minimize scarring.

The present disclosure is also directed to providing a cosmetic mask pack that can promote wound healing and minimize scarring.

### Technical Solution

A hydrogel patch for promoting wound healing or minimizing scarring of the present disclosure contains a biocompatible polymer modified with drug-loaded pyrogallol,
wherein the drug may be a single drug consisting of a polypeptide in which a polypeptide containing an amino acid sequence of SEQ ID NO: 1 is fused to a polypeptide containing an amino acid sequence encoding a PTD (protein transduction domain), or a combination drug of the fused polypeptide and valproic acid.

In the combination drug, the polypeptide in which the polypeptide containing an amino acid sequence of SEQ ID NO: 1 is fused to the polypeptide containing an amino acid sequence encoding a PTD (protein transduction domain), and the valproic acid may be mixed at a molar ratio of 1:100-1000.

The single drug or the combination drug may be loaded at a concentration of 0.3 to 10 mg/mL.

The content of collagen III at a wound site may be increased by the hydrogel patch, such that the ratio of collagen III/collagen I may be from 2 to 70.

The hydrogel patch may enhance the expression of the dermal stem cell marker CD105 and the angiogenesis marker CD31 at the wound site.

The biocompatible polymer may be selected from a group consisting of hyaluronic acid, heparin, cellulose, dextran, alginate, chitosan, chitin, collagen, gelatin, chondroitin sulfate, and pectin.

The biocompatible polymer may be hyaluronic acid.

The PTD (protein transduction domain) may include poly R8, HIV-Tat, HSV VP22, Antp, or transportan.

The polypeptide may further contain a linker inserted between the amino acid sequence of SEQ ID NO: 1 and the amino acid sequence encoding the PTD.

The hydrogel patch may be a patch for a medical device.

The cosmetic mask pack for promoting wound healing or minimizing scarring of the present disclosure includes a hydrogel patch containing a biocompatible polymer modified with drug-loaded pyrogallol, wherein the drug may be a single drug consisting of a polypeptide in which a polypeptide containing an amino acid sequence of SEQ ID NO: 1 is fused to a polypeptide containing an amino acid sequence encoding a PTD (protein transduction domain), or a combination drug combination drug of the fused polypeptide and valproic acid.

### Advantageous Effects

Although wound healing is promoted and scar formation is alleviated by PTD-DBM (single drug) or a combination drug consisting of PTD-DBM and VPA, a hydrogel patch of the present disclosure in which each of the single drug or the combination drug combination drug is loaded in HA-PG shows a better effect of wound healing promotion and alleviating scar formation reduction than the single drug or combination drug combination drug alone.

Furthermore, when a hydrogel patch containing the single drug or combination drug of the present disclosure is applied to a wound site, the content of collagen III increases, which is a pattern observed in fetal wound healing where wounds heal without scarring, indicating that scarring can be minimized.

Furthermore, when a hydrogel patch containing the single drug or combination drug of the present disclosure is applied to a wound site, wound healing can be promoted and scarring can be minimized since the dermal stem cell marker CD105 and the angiogenesis marker CD31 are increased.

The hydrogel patch containing the single drug or combination drug of the present disclosure can be used not only for a medical device but also for a cosmetic mask pack.

### BRIEF DESCRIPTION OF DRAWINGS

FIG. 1A shows photographs showing the scar area on day 0 (0 d), day 3 (3 d), day 6 (6 d), day 9 (9 d), and day 11 (11 d) for a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups; and FIG. 1B shows a result of quantifying the scar area of each group on day 11 of wound healing using the Image J program.

FIG. 2A shows the H&E staining images of a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups on day 11 of wound healing; and FIG. 2B shows a result of measuring scar width (a.u.) in wound tissue for each group on day 11 of wound healing.

FIG. 3A shows the immunohistochemical staining images for collagen I and collagen III of a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups on day 11 of wound healing; and FIG. 3B shows a result of measuring the average intensity of collagen I and collagen III for each group, and quantifying the ratio of collagen III/collagen I.

FIG. 4A shows the immunohistochemical staining images for CD105 in the wound dermis of a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups for on day 11 of wound treatment; and FIG. 4B shows a result of quantifying the number of CD105-positive cells in the wound dermis for each group.

FIG. 5A shows the immunohistochemical staining images for CD31, which is a marker of angiogenesis, on day 11 of wound healing for a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups; and FIG. 5B shows a result of quantifying the number of CD31-positive cells for each group.

### BEST MODE

The present disclosure relates to a therapeutic hydrogel patch that can promote wound healing and minimize scarring, and a cosmetic mask pack including the same.

The hydrogel patch refers to a thin membrane-like structure having a predetermined thickness and made of a biocompatible polymer, which can be cut into a desired shape.

The present disclosure will now be described in detail.

The hydrogel patch for promoting wound healing or minimizing scarring of the present disclosure contains a biocompatible polymer modified with drug-loaded pyrogallol, wherein the drug may be a single drug consisting of a polypeptide in which a polypeptide containing an amino acid sequence of SEQ ID NO: 1 is fused to a polypeptide containing an amino acid sequence encoding a PTD (protein transduction domain), or a combination drug of the fused polypeptide and valproic acid.

### Pyrogallol-Modified Biocompatible Polymer (HA-PG)

The pyrogallol is 1,2,3-trihydroxybenzene with three adjacent hydroxyl (—OH) groups. When oxidized, it forms covalent crosslinking with various functional groups. In particular, because it can be rapidly oxidized naturally within several minutes in an environment of high oxygen concentration, such as in the human body, the hydrogel patch can be advantageously applied directly without any oxidant treatment in clinical practice.

The biocompatible polymer may be selected from a group consisting of hyaluronic acid, heparin, cellulose, dextran, alginate, chitosan, chitin, collagen, gelatin, chondroitin sulfate, and pectin. Specifically, it may be hyaluronic acid.

Since the hydrogel patch containing the pyrogallol-modified biocompatible polymer (hereinafter, referred to as a hydrogel patch) has a nanofiber-based porous structure and is more swellable as compared to a solution-based hydrogel containing a pyrogallol-modified biocompatible polymer (hereinafter, referred to as a bulk hydrogel), it can effectively load cells or drugs within the patch in an in-vivo environment. In addition, since the hydrogel patch has better elasticity, mechanical properties, and tissue adhesivity than the bulk hydrogel, it can effectively deliver drugs to tissues in vivo.

The hydrogel patch of the present disclosure may have a thickness of 0.05 to 10.0 mm, specifically 0.1 to 5.0 mm. If the thickness of the hydrogel patch is below the lower limit, the durability and adhesivity may be reduced after drug loading, and if it exceeds the upper limit, drug release time frame may be prolonged.

### Single Drug (PTD-DBM) or Combination Drug (Mixture of PTD-DBM and Valproic Acid)

The drug of the present disclosure may be a single drug consisting of a polypeptide (PTD-DBM) in which a polypeptide containing an amino acid sequence of SEQ ID NO: 1 is fused to a polypeptide containing an amino acid sequence encoding a PTD (protein transduction domain), or a combination drug of the PTD-DBM and valproic acid (VPA).

The PTD-DBM (protein transduction domain-fused Dvl-binding motif) is a peptide that interferes with the CXXC5-disheveled (Dvl) interaction.

[SEQ ID NO: 1]:
RKTGHQICKFRKC

The amino acid sequence of SEQ ID NO: 1 is a DBM peptide (Dvl minimal binding peptide) that corresponds to a portion of the amino acid sequences of the proteins CXXC4 and CXXC5 (CXXC-type zinc finger protein 5). CXXC5 binds to Dvl (disheveled), thereby blocking signal transduction by the Wnt/β-catenin signaling system. In the present disclosure, the Wnt/β-catenin signaling system is activated by blocking the interaction of CXXC5 with Dvl by the DBM peptide.

In addition, the amino acid sequence encoding the PTD (protein transduction domain) may be further inserted into the amino acid sequence of SEQ ID NO: 1 in order to allow the peptide (DBM) of the amino acid sequence of SEQ ID NO: 1 to induce intracellular penetration through the cell membrane.

The PTD is mainly composed of basic amino acids such as arginine, lysine, etc. The penetration of the PTD into a cell may be induced by adding the PTD to the N-terminal or C-terminal of the amino acid sequence of SEQ ID NO: 1.

The PTD may be poly R8, HIV-Tat, HSV VP22, Antp, or transportan, wherein the poly R8 may have an amino acid sequence of SEQ ID NO: 2, the HIV-Tat may have an amino acid sequence of SEQ ID NO: 3, the HSV VP22 may have an amino acid sequence of SEQ ID NO: 4, the Antp may have an amino acid sequence of SEQ ID NO: 5, or the transportan may have an amino acid sequence of SEQ ID NO: 6.

[SEQ ID NO: 2]:
RRRRRRRR
[SEQ ID NO: 3]:
YGRKKRRQRRR
[SEQ ID NO: 4]:
DAATATRGRSAASRPTERPRAPARSASRPRRPVE
[SEQ ID NO: 5]:
RQIKIWFQNRRMKWKK
[SEQ ID NO: 6]:
AGYLLGKINLKALAALAKKIL

Furthermore, the polypeptide according to the present disclosure may further contain a linker inserted between the amino acid sequence of SEQ ID NO: 1 and the amino acid sequence encoding the PTD. A specific example of the linker inserted between the amino acid sequence of SEQ ID NO: 1 and the amino acid sequence encoding the PTD is a sequence of 4 to 12 amino acids, which may be 4 to 8 glycines, and the polypeptide with the linker further inserted may have the amino acid sequence of SEQ ID NO: 7.

[SEQ ID NO: 7]:
RRRRRRRRGGGGRKTGHQICKFRKC

Furthermore, the valproic acid (VPA), which is used as a GSK3β (glycogen synthase kinase 3β) inhibitor, may further promote wound healing and reduce scar formation when used in combination with PTD-DBM.

The PTD-DBM and valproic acid may be mixed at a molar ratio of 1:100-1000, specifically 1:200-500. If the content of the valproic acid based on the PTD-DBM is below the lower limit, the ratio of collagen III/collagen I may decrease. And, if it exceeds the upper limit, the CD105 marker and the CD31 marker may not increase.

The single drug or the combination drug of the present disclosure can be loaded into the hydrogel patch at a concentration of 0.3 to 10 mg/mL, specifically 1 to 8 mg/mL. If the concentration of the combination drug to be loaded is below the lower limit, the effect may be insignificant. And, if it exceeds the upper limit, the effect of scar reduction may decrease.

When the hydrogel patch containing the single drug or the combination drug of the present disclosure is applied to a wound site, the content of collagen III, which is important for regenerative wound healing, is increased at the wound site. As a result, the ratio of collagen III/collagen I is increased up to 2 to 70, specifically 15 to 65, more specifically 52 to 60, and therefore, scarring can be minimized.

Furthermore, the application of the hydrogel patch containing the single drug or the combination drug of the present disclosure to a wound site can increase the dermal stem cell marker CD105 and the angiogenesis marker CD31, thereby promoting wound healing and minimizing scarring.

The hydrogel patch containing the single drug or the combination drug of the present disclosure can be used not only for a medical device but also for a cosmetic mask pack.

### MODE FOR INVENTION

### Preparation Example 1. Hydrogel (HA-PG)

A hydrogel (HA-PG) was synthesized via carbodiimide chemistry.

Hyaluronic acid (HA, molecular weight 200 kDa, Lifecore Biomedical, Chaska, MN, USA) was completely dissolved in triple-distilled water (TDW) to a concentration of 10 mg/mL, and then stirred for 15 minutes after adding 3-ethylcarbodiimide hydrochloride (EDC, Thermo Fisher Scientific, Waltham, MA, USA), such that the molar ratio of the hyaluronic acid was 1:1.5. Then, after adding N-hydroxysuccinimide (NHS, Sigma-Aldrich, St. Louis, MO, USA) such that the molar ratio of the hyaluronic acid and NHS was 1:1, the mixed solution was stirred further at pH 5.5 for 15 minutes to activate the carboxyl group of the hyaluronic acid, 5-hydroxydopamine hydrochloride (Sigma-Aldrich) was added to the reaction solution such that the molar ratio of the hyaluronic acid and the 5-hydroxydopamine hydrochloride was 1:1. The mixture was stirred at pH 4.5-5.0 for 24 hours to obtain HA-PG.

The unreacted chemical substances and byproducts were removed by dialysis using a 6-8 kDa blocking membrane (Membrane Filtration Products Inc., Seguin, TX, USA) against acidic PBS (Bioscience, Seongnam, Gyeonggi, Korea), and the obtained HA-PG was lyophilized and stored at 4° C. until use. The successful synthesis of HA-PG was confirmed by 1H-NMR spectroscopy at 300 MHz (Bruker, Billerica, MA, USA).

### Example 1. PTD-DBM Alone

To test the efficacy of a polypeptide containing the amino acid sequence of SEQ ID NO: 1, a PolyR8-DBM peptide, wherein poly R8 (RRRRRRRR) and a polyglycine linker (GGGG) were attached at the amino acid terminus of SEQ ID NO: 1, and lysine and the fluorescent dye fluorescein isothiocyanate (FITC) were attached at the carboxyl terminus, was synthesized.

PolyR8-DBM:
RRRRRRRRGGGGRKTGHQICKFRKCK-(FITC)

### Example 2. Combination Drug (PTD-DBM+VPA)

### PTD-DBM

To test the efficacy of a polypeptide containing the amino acid sequence of SEQ ID NO: 1, a PolyR8-DBM peptide, which contained poly R8 (RRRRRRRRR) and a polyglycine linker (GGGG) at the amino acid terminus of SEQ ID NO: 1, plus lysine and the fluorescent dye FITC (fluorescein isothiocyanate) at the carboxyl terminus, was synthesized.

PolyR8-DBM:
RRRRRRRRGGGGRKTGHQICKFRKCK-(FITC)

### Combination Drug

A combination drug was obtained by mixing the PolyR8-DBM peptide (PTD-DBM) and valproic acid at a molar ratio of 1:500.

### Comparative Example 1. VPA Alone

Valproic acid was used alone.

### Comparative Example 2. EGF Alone

Epidermal growth factor (EGF) was used to promote wound healing.

### Test Examples

A control group was an untreated group.

In the attached drawings, Topical refers to the topical application of the drugs prepared in the examples and comparative examples to mice, and Patch refers to hydrogel patches obtained by loading the drugs prepared in the examples and comparative examples in the HA-PG prepared in Example 1.

Specifically, the hydrogel patches were obtained by dissolving the HA-PG prepared in Example 1 in PBS to a concentration of 1% (w/v), pouring the HA-PG solution loaded with each of the drugs prepared in the examples and comparative examples into a pre-designed 1 cm×1 cm rectangular mold, and then freeze-drying the same completely to obtain each HA-PG patch. The prepared HA-PG patches were stored at −20° C. before use to avoid moisture.

### Animal Experiment

6-week-old male C3H mice weighing 21-26 g, obtained from Coatech, were acclimatized to a new environment for 1 week for use in measuring the effect of the hydrogel patches loaded with the drugs prepared in the examples and comparative examples on wound healing and alleviation of scar formation. They were housed in acrylic cages (45×60×25 cm) with free access to sufficient food and water and a 12-hour light/dark cycle (lights on from 8:00 am). They were kept under the condition of constant temperature (20-24° C.) and humidity (45-65%). After 1 week of acclimatization to the new environment, it was investigated whether any abnormal behavior was observed while maintaining the sleep cycle.

7-week-old C3H mice with telogen hair follicles were anesthetized with 2,2,2-tribromoethanol (Sigma Aldrich). After shaving the hair on the back of the mouse with clippers, a wound with a size of 1 cm² was created on the back of the mice. For the topical treatment groups (Topical), the drugs were applied daily to the wound every day. And, for the hydrogel patch groups (Patch), the hydrogel patches were applied once to the wound.

TABLE 1
Mice of 10 groups

| | |
|---|---|
| Topical | Control: phosphate-buffered saline |
| (applied | PTD-DBM: 100 μM of of PTD-DBM of Example 1 applied every day |
| every day) | VPA: 50 mM of VPA of Comparative Example 1 applied every day |
| | PTD-DBM + VPA: A combination drug of 100 μM of PTD-DBM and |
| | 50 mM of VPA of Example 2 applied every day |
| | EGF: 100 μM of EGF of Comparative Example 2 applied every day |
| Patch | Control: phosphate-buffered saline |
| (attached | PTD-DBM: PTD-DBM of Example 1 loaded at a concentration of |
| once) | 0.3 mg/mL |
| | VPA: VPA of Comparative Example 1 loaded at a concentration of |
| | 7.2 mg/mL |
| | PTD-DBM + VPA: combination drug of 0.3 mg/mL PTD-DBM and |
| | 7.2 mg/mL VPA of Example 2 |
| | EGF: EGF of Comparative Example 2 loaded at a concentration of |
| | 0.6 mg/mL |

Data are presented as mean±SD. Statistical analysis was performed using the unpaired two-tailed Student's t-test. Statistical significance is presented as *P<0.05, **P<0.005, ***P<0.0005.

### Test Example 1. Measurement of Wound Area of Mice

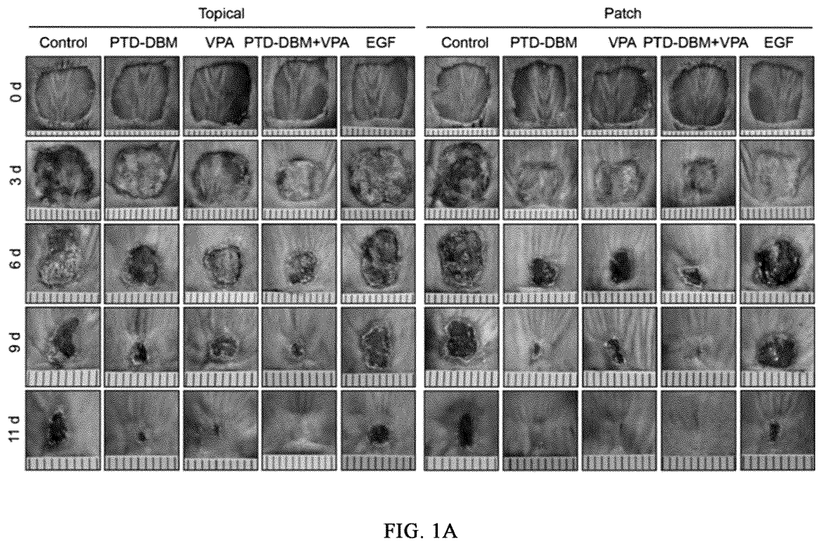

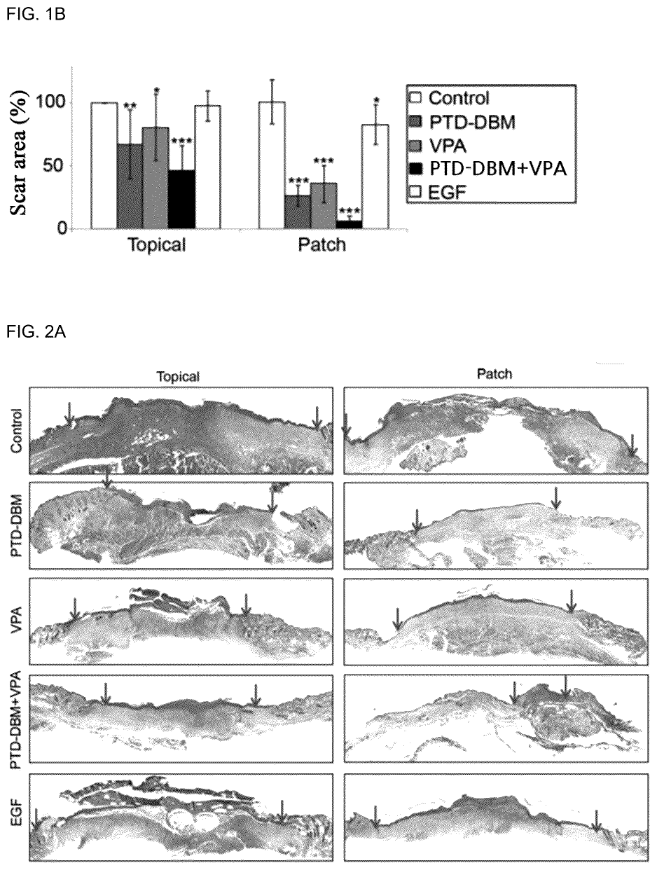

FIG. 1A shows photographs showing the scar area on day 0 (0 d), day 3 (3 d), day 6 (6 d), day 9 (9 d), and day 11 (11 d) for a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups; and FIG. 1B shows a result of quantifying the scar area of each group on day 11 of wound healing using the Image J program.

As shown in FIG. 1A and FIG. 1B, in both the Topical and Patch groups, it was found that the PTD-DBM of Example 1 and the PTD-DBM combined with VPA of Example 2, promoted wound healing and reduced scar formation significantly as compared to the other groups.

Furthermore, the HA-PG patches loaded with the drugs of Example 1, Example 2, Comparative Example 1, and Comparative Example 2 showed better effect of healing promotion wound and reducing scar formation than topical application (Topical). Among them, the PTD-DBM+VPA_HA-PG patch group loaded with the combination drug of PTD-DBM and VPA of Example 2 showed the best effect of healing promotion wound and reducing scar formation.

### Test Example 2. Hematoxylin and Eosin (H&E) Staining

Mouse wound tissues were fixed overnight at 4° C. in 4% (w/v) paraformaldehyde (PFA), dehydrated and then embedded in paraffin, and sectioned to 4 μm thickness. The sections were deparaffinized and rehydrated using xylene and ethanol. The slides were incubated with Harris hematoxylin for 5 minutes and with eosin for 1 minute. The H&E-stained slides were observed using a bright-field light microscope (ECLIPSE TE2000-U, Nikon).

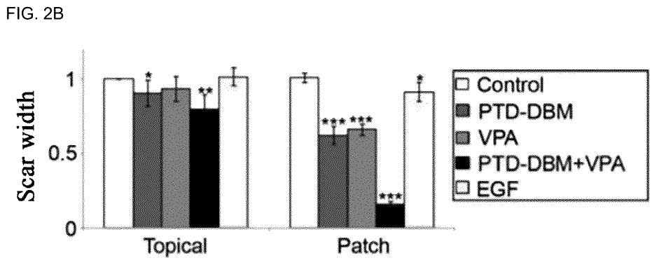

FIG. 2A shows the H&E staining images of a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups on day 11 of wound healing; and FIG. 2B shows a result of measuring scar width (a.u.) in wound tissue for each group on day 11 of wound healing.

As shown in FIG. 2A and FIG. 2B, in both the Topical and Patch groups, it was found that the PTD-DBM of Example 1 and the PTD-DBM combined with VPA of Example 2 promoted wound healing and reduced scar formation significantly as compared to the other groups.

Furthermore, the HA-PG patches loaded with the substances of Example 1, Example 2, Comparative Example 1, and Comparative Example 2 showed better effect of healing promotion wound and reducing scar formation than topical application. Among them, the PTD-DBM+VPA_HA-PG patch group loaded with the combination drug of PTD-DBM and VPA of Example 2 showed significantly better effect of healing promotion wound and reducing scar formation.

### Test Example 3. Immunohistochemical Staining

After deparaffinizing and rehydrating 4 μm-thick paraffin sections, antigens were retrieved using 10 mM sodium citrate buffer. After cooling, the slides were incubated in PBS, blocked with 5% bovine serum albumin (BSA) in PBS for 30 minutes at room temperature, and then the incubated with a dilution solution of primary antibodies overnight at 4° C.

After washing anti-collagen I (1:200, Abcam), anti-collagen III (1:200, Novus Biologicals), anti-CD105 (1:50, R&D systems), and anti-CD31 (1:100, Abcam) antibodies with PBS, the slides were incubated with Alexa Fluor 488- or Alexa Fluor 555-conjugated IgG secondary antibodies (Invitrogen, 1:500) for 1 hour at room temperature and then stained with DAPI (Boehringer Mannheim, 1:5000) for 10 minutes. The stained images were examined using a Nikon Eclipse Ti microscope (Nikon).

Quantification was performed using the NIS Elements V3.2 software (Nikon).

### 3-1. Immunohistochemical Staining for Collagen I and Collagen III

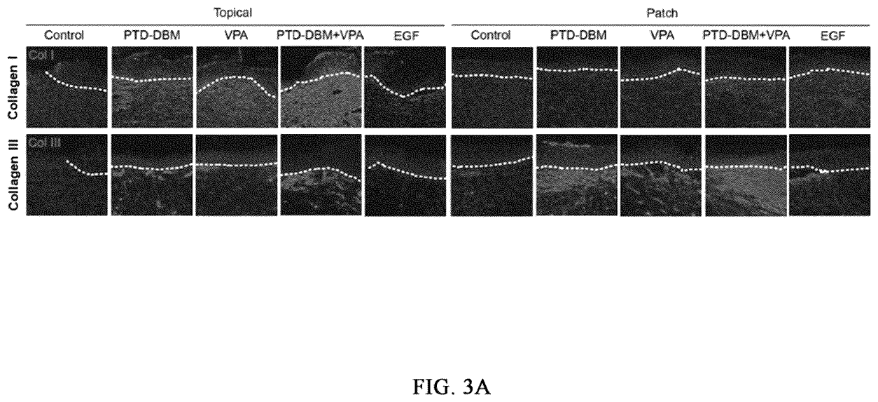

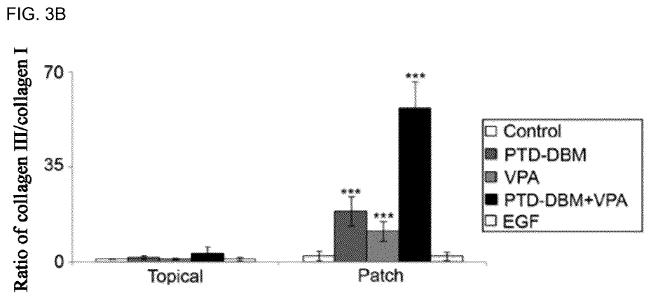

FIG. 3A shows the immunohistochemical staining images for collagen I and collagen III of a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups on day 11 of wound healing; and FIG. 3B shows a result of measuring the average intensity of collagen I and collagen III for each group, and quantifying the ratio of collagen III/collagen I.

As shown in FIG. 3A, among the Topical groups, the control group, the PTD-DBM group, the VPA group, the PTD-DBM+VPA group, and the EGF group showed an increase in collagen I. However, the control group, the PTD-DBM group, the VPA group, the PTD-DBM+VPA group, and the EGF group in the Patch groups showed an increase in collagen III as compared to the respective groups of the Topical groups.

Furthermore, as shown in FIG. 3B, among the Topical groups, the control group, the PTD-DBM group, the VPA group, the PTD-DBM+VPA group, and the EGF group showed a very low collagen III/collagen I ratio of 10 or lower. However, the control group, the PTD-DBM group, the VPA group, the PTD-DBM+VPA group, and the EGF group in the Patch groups showed higher collagen III/collagen I ratios as compared to the respective groups of the Topical groups.

In particular, among the Patch groups, the PTD-DBM+VPA_HA-PG patch group loaded with the combination drug of PTD-DBM and VPA of Example 2 showed a significantly higher collagen III/collagen I ratio of 57.2.

### 3-2. Immunohistochemical Staining for CD105 in Wound Dermis

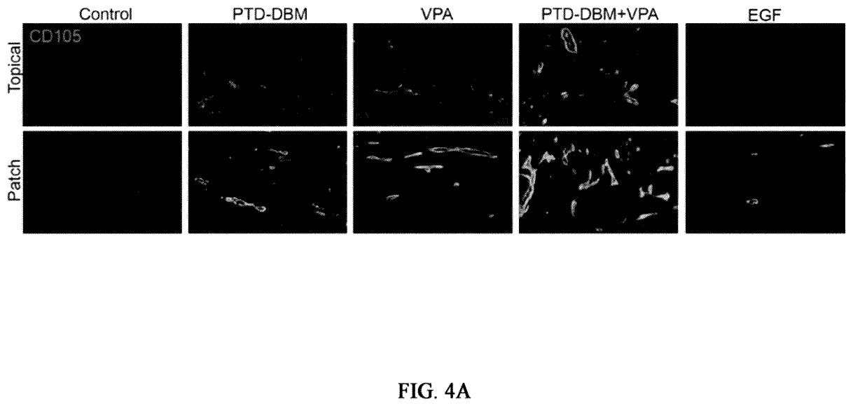

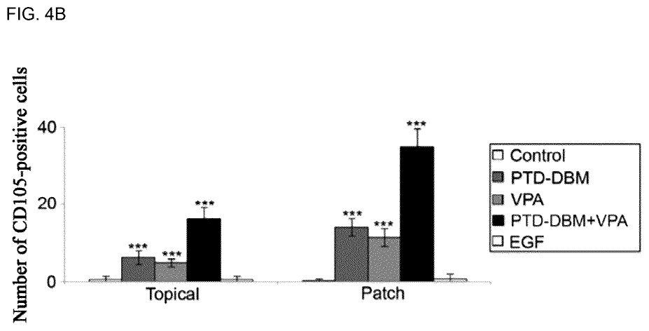

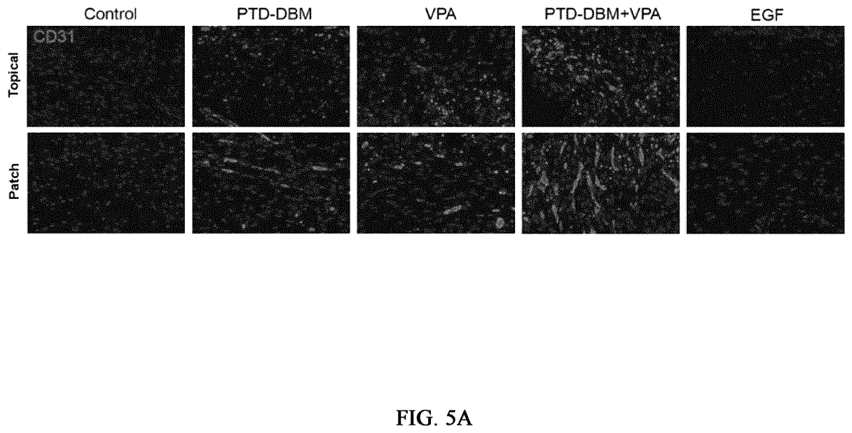

FIG. 4A shows the immunohistochemical staining images for CD105 in the wound dermis of a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups for on day 11 of wound treatment; and FIG. 4B shows a result of quantifying the number of CD105-positive cells in the wound dermis for each group.

As shown in FIG. 4A and FIG. 4B, in both the Topical and Patch groups, the PTD-DBM of Example 1 and the PTD-DBM combined with and VPA of Example 2 showed significant increase in the stem cell marker CD105 in the wound dermis as compared to the other groups.

Furthermore, the HA-PG patches loaded with the substances of Example 1, Example 2, Comparative Example 1, and Comparative Example 2 showed more increase in CD105 than when they were applied topically. Among them, the PTD-DBM+VPA_HA-PG patch group loaded with the combination drug of PTD-DBM and VPA of Example 2 showed a significantly increased number of CD105-positive cells.

### 3-3. Immunohistochemical Staining for CD31

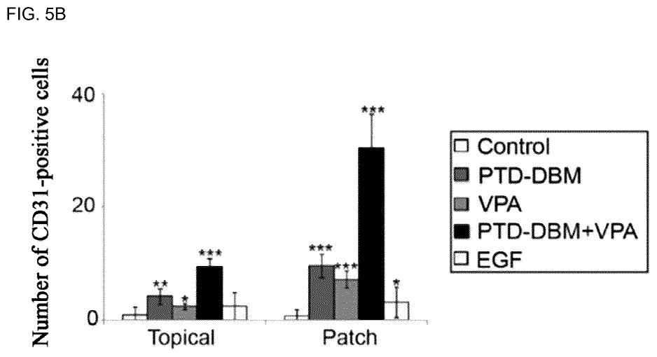

FIG. 5A shows the immunohistochemical staining images for CD31, which is a marker of angiogenesis, on day 11 of wound healing for a control group, a PTD-DBM group, a VPA group, a PTD-DBM+VPA group, and an EGF group as Topical groups; and a control group, a PTD-DBM group, a VPA group, a PTD-DBM+VPA group, and an EGF group as Patch groups; and FIG. 5B shows a result of quantifying the number of CD31-positive cells for each group.

As shown in FIG. 5A and FIG. 5B, in both the Topical and Patch groups, the PTD-DBM of Example 1 and the PTD-DBM combined with and VPA of Example 2 showed significant increase in the angiogenesis marker CD31 as compared to the other groups.

Furthermore, the HA-PG patches loaded with the substances of Example 1, Example 2, Comparative Example 1, and Comparative Example 2 showed more increase in CD31 than when they were applied topically. Among them, the PTD-DBM+VPA_HA-PG patch group loaded with the combination drug of PTD-DBM and VPA of Example 2 showed a significantly increased number of CD31-positive cells.

### INDUSTRIAL APPLICABILITY

The hydrogel patch containing the single drug or combination drug of the present disclosure can be used not only for a medical device but also for a cosmetic mask pack.

[Sequence List Pretext]
[SEQ ID NO: 1]:
RKTGHQICKFRKC
[SEQ ID NO: 2]:
RRRRRRRR
[SEQ ID NO: 3]:
YGRKKRRQRRR
[SEQ ID NO: 4]:
DAATATRGRSAASRPTERPRAPARSASRPRRPVE
[SEQ ID NO: 5]:
RQIKIWFQNRRMKWKK
[SEQ ID NO: 6]:
AGYLLGKINLKALAALAKKIL
[SEQ ID NO: 7]:
RRRRRRRRGGGGRKTGHQICKFRKC

1. A hydrogel patch for promoting wound healing or minimizing scarring, comprising a biocompatible polymer modified with drug-loaded pyrogallol, wherein the drug is a single polypeptide drug in which a polypeptide consisting of an amino acid sequence of SEQ ID NO: 1 is fused to a polypeptide consisting of an amino acid sequence encoding a PTD (protein transduction domain), or a combination drug consisting of a polypeptide in which a polypeptide consisting of an amino acid sequence of SEQ ID NO: 1 is fused to a polypeptide consisting of an amino acid sequence encoding a PTD (protein transduction domain), and valproic acid.

2. The hydrogel patch for promoting wound healing or minimizing scarring according to claim 1, wherein the combination drug is a mixture of a polypeptide in which a polypeptide containing an amino acid sequence of SEQ ID NO: 1 is fused to a polypeptide containing an amino acid sequence encoding a PTD (protein transduction domain) and valproic acid at a molar ratio of 1:100-1000.

3. The hydrogel patch for promoting wound healing or minimizing scarring according to claim 1, wherein the hydrogel patch enhances the expression of the dermal stem cell marker CD105 and the angiogenesis marker CD31 at a wound site.

4. The hydrogel patch for promoting wound healing or minimizing scarring according to claim 1, wherein the biocompatible polymer is selected from a group consisting of hyaluronic acid, heparin, cellulose, dextran, alginate, chitosan, chitin, collagen, gelatin, chondroitin sulfate, and pectin.

5. The hydrogel patch for promoting wound healing or minimizing scarring according to claim 1, wherein the PTD (protein transduction domain) comprises poly R8, HIV-Tat, HSV VP22, Antp, or transportan.

6. The hydrogel patch for promoting wound healing or minimizing scarring according to claim 1, wherein the polypeptide further comprises a linker inserted between the amino acid sequence of SEQ ID NO: 1 and the amino acid sequence encoding the PTD.

7. The hydrogel patch for promoting wound healing or minimizing scarring according to claim 1, wherein the hydrogel patch is a patch for a medical device.

8. A cosmetic mask pack for promoting wound healing or minimizing scarring, comprising a hydrogel patch comprising a biocompatible polymer modified with drug-loaded pyrogallol, wherein the drug is a single polypeptide drug in which a polypeptide consisting of an amino acid sequence of SEQ ID NO: 1 is fused to a polypeptide consisting of an amino acid sequence encoding a PTD (protein transduction domain), or a combination drug consisting of a polypeptide in which a polypeptide consisting of an amino acid sequence of SEQ ID NO: 1 is fused to a polypeptide consisting of an amino acid sequence encoding a PTD (protein transduction domain), and valproic acid.

9. The cosmetic mask pack for promoting wound healing or minimizing scarring according to claim 8, wherein the combination drug is a mixture of a polypeptide in which a polypeptide containing an amino acid sequence of SEQ ID NO: 1 is fused to a polypeptide containing an amino acid sequence encoding a PTD (protein transduction domain) and valproic acid at a molar ratio of 1:100-1000.

10. The cosmetic mask pack for promoting wound healing or minimizing scarring according to claim 8, wherein the hydrogel patch enhances the expression of the dermal stem cell marker CD105 and the angiogenesis marker CD31 at a wound site.

11. The cosmetic mask pack for promoting wound healing or minimizing scarring according to claim 8, wherein the biocompatible polymer is selected from a group consisting of hyaluronic acid, heparin, cellulose, dextran, alginate, chitosan, chitin, collagen, gelatin, chondroitin sulfate, and pectin.

12. The cosmetic mask pack for promoting wound healing or minimizing scarring according to claim 8, wherein the PTD (protein transduction domain) comprises poly R8, HIV-Tat, HSV VP22, Antp, or transportan.

13. The cosmetic mask pack for promoting wound healing or minimizing scarring according to claim 8, wherein the polypeptide further comprises a linker inserted between the amino acid sequence of SEQ ID NO: 1 and the amino acid sequence encoding the PTD.


SEQUENCE LISTING (7 sequences)

SEQ ID NO: 1 - protein, 39 aa, synthetic construct
ARGLYSTHRGLYHISGLNILECYSLYSPHEARGLYSCYS

SEQ ID NO: 2 - protein, 24 aa, synthetic construct
ARGARGARGARGARGARGARGARG

SEQ ID NO: 3 - protein, 33 aa, synthetic construct
TYRGLYARGLYSLYSARGARGGLNARGARGARG

SEQ ID NO: 4 - protein, 48 aa, synthetic construct
ASPALAALATHRALATHRARGGLYARGSERALAALASERARGPROTHR

SEQ ID NO: 5 - protein, 48 aa, synthetic construct
ARGGLNILELYSILETRPPHEGLNASNARGARGMETLYSTRPLYSLYS

SEQ ID NO: 6 - protein, 48 aa, synthetic construct
ALAGLYTYRLEULEUGLYLYSILEASNLEULYSALALEUALAALALEU

SEQ ID NO: 7 - protein, 75 aa, synthetic construct
ARGARGARGARGARGARGARGARGGLYGLYGLYGLYARGLYSTHRGLYHISGLNILECYSLYSPHEARGLYSCYS